(19) United States
(12) Patent Application Publication
(10) Pub. No.: US 2015/0018649 A1
(43) Pub. Date: Jan. 15, 2015

(54) METHODS AND SYSTEMS FOR USING A DIFFERENTIAL LIGHT DRIVE IN A PHYSIOLOGICAL MONITOR

(71) Applicant: Covidien LP, Mansfield, MA (US)

(72) Inventors: Daniel Lisogurski, Boulder, CO (US); Christopher Meehan, Golden, CO (US); William Kit Dean, Castle Pines, CO (US); Andy S. Lin, Boulder, CO (US)

(21) Appl. No.: 13/942,391

(22) Filed: Jul. 15, 2013

(51) Int. Cl. H05B 33/08; A61B 5/1455; A61B 5/00

(52) U.S. Cl. CPC H05B 33/0842; A61B 5/0082; A61B 5/14552
USPC 600/323; 315/307; 315/297; 600/476

(57) ABSTRACT

A physiological monitoring system may use a differential light drive to illuminate one or more light sources. A differential light drive may include applying two signals, one to each terminal of a light emitting diode or other light source, such that the illumination of the light source is controlled by the difference between the two light drive signals. In some embodiments, light emitting diodes may be turned on and off using a differential light drive without using switches and using only unipolar voltage sources. In some embodiments, light drive signals may be 180 degrees out-of-phase, and the phase shift may be used to reduce crosstalk and other electronic noise, for example by carrying the signals in a twisted pair of conductors.

The present disclosure relates to operating a physiological monitor, and more particularly relates to using a differential light drive in a pulse oximeter or other medical device.

### SUMMARY

Methods and systems are provided for using a differential light drive in a physiological monitor. In some embodiments, a first light drive signal is provided to a first terminal of a light emitting device and a second light drive signal is provided to a second terminal. The voltage across the device corresponds to the voltage difference between the two signals. In some embodiments, for example where the light emitters are light emitting diodes, this may allow the illumination of a light emitter to be controlled without using switching and using only a positive supply rail and/or unipolar power supplies. In some embodiments, noise and crosstalk may be reduced, for example by carrying 180 degree out-of-phase signals on respective conductors of a twisted pair of wires.

In some embodiments, two light emitters may be connected to three signal generators, such that the first and second signal generators provide a first differential light drive signal to a first light emitter, and the third and second signal generators provide a second differential light drive signal to the second emitter. In some embodiments, the second light drive signal may be a constant bias voltage, while the first and third light drive signals are periodic signals. The first and second differential light drive signals may be used to control the illumination of the first and second light emitters, respectively. The periodic signals may be 180 degrees out-of-phase with one another and carried in adjacent conductors, such that electromagnetic signal noise is cancelled.

In some embodiments, two light emitters are wired in a back-to-back configuration, for example, two light emitting diodes (LEDs) may be wired in an opposite parallel configuration. The cathode of a first LED and the anode of a second LED may be connected to a first light drive signal generator, and the anode of the first LED and the cathode of the second LED may be connected to a second light drive signal generator. In some embodiments, a light drive current may flow through the first LED when the voltage at the first light drive signal generator is higher than the voltage at the second light drive signal generator, and may flow through the second LED when the voltage at the first light drive signal generator is lower than the voltage at the second light drive signal generator. Thus, where the currents in the connections are 180 degrees out-of-phase, the two LEDs may be alternately illuminated.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 is a block diagram of an illustrative physiological monitoring system in accordance with some embodiments of the present disclosure;

FIG. 2A shows an illustrative plot of a light drive signal in accordance with some embodiments of the present disclosure;

FIG. 2B shows an illustrative plot of a detector signal that may be generated by a sensor in accordance with some embodiments of the present disclosure;

FIG. 3 is a perspective view of an embodiment of a physiological monitoring system in accordance with some embodiments of the present disclosure;

FIG. 4 shows an illustrative circuit diagram including two AC voltage sources in a differential light drive in accordance with some embodiments of the present disclosure;

FIG. 5 shows illustrative signal plots corresponding to using two AC voltage sources in a differential light drive in accordance with some embodiments of the present disclosure;

FIG. 6 shows an illustrative circuit diagram including one AC voltage source in a differential light drive in accordance with some embodiments of the present disclosure;

FIG. 7 shows illustrative signal plots corresponding to using one AC voltage source in a differential light drive in accordance with some embodiments of the present disclosure; and

FIG. 8 shows an illustrative flow diagram including steps for using a differential light drive in accordance with some embodiments of the present disclosure.

### DETAILED DESCRIPTION OF THE FIGURES

The present disclosure is directed towards using a differential light drive in a medical device. A differential light drive may use two light drive generators to drive a light emitter, such that the voltage applied to the emitter is the difference between the voltage provided by the two respective generators. In some embodiments, a medical device may include optical devices and may use a differential light drive to reduce noise, crosstalk, and interference. The use of a differential light drive may reduce the need for electronic noise shielding and may improve performance, for example by reducing signal errors. In some embodiments, a differential light drive may be provided by applying modulation to two or more light drive generators.

Light emitters may be used to generate light signals. A light emitter may include a two terminal device such as a light emitting diode. In some embodiments, a differential light drive may be used to apply voltages to each terminal of a light emitter such that the light emission may be turned on and off without using switches. In some embodiments, this technique may reduce noise associated with switching, may reduce cross-talk between wires, and may reduce other undesired signal components.

Light emitting diodes may be used as light emitters in a medical device. Light emitting diodes (LEDs) include solid state devices that allow current to flow in only one direction, and emit light when the proper currents and voltages are applied to the terminals of the diode. Emitted light may be of any suitable wavelength, for example, red or infrared. LEDs may include an anode and a cathode. As referred to herein, a diode generally allows positive currents to flow from the anode to the cathode, and blocks current flowing from the cathode to the anode. When the voltage difference between the anode and the cathode is above a particular positive threshold, and a suitable current is flowing through the diode, light is emitted. This threshold may be referred to as the turn-on voltage. In an example, a diode may emit light when the voltage difference between the anode and cathode is 3V, and when the current flowing through the diode is between 1 mA and 10 mA. The amount of current flowing through the LED when it is in an on state may change the amount of light emitted from the LED, that is, brightness. It will be understood that, as used here, an on state of an LED is when light is being emitted from the LED, and an off state is when light is not being emitted.

A differential drive, as used herein, refers to applying voltage and/or current sources to terminals of a device with at least two inputs, such that the voltage and/or current provided to the device is determined by the difference between the two sources. In some embodiments, using a differential light drive may allow LEDs to be switched on and off using only a positive power supply, that is, a unipolar supply, although bipolar supplies may also be used. As used herein, a unipolar supply provides an output of only a single polarity. For example, a unipolar voltage supply may only supply positive voltages. In an example, an AC signal may be applied to one terminal of an LED and a DC bias signal may be applied to the second terminal of an LED. When the differential voltage across the LED is above the turn-on voltage of the device, light is emitted. When the differential voltage falls below the threshold, the LED does not emit light. Thus by adjusting the level of the DC bias signal, the LED may be turned on or off.

Crosstalk occurs when a signal transmitted in a first channel creates an undesired effect in another channel. In conductive signal transmission, crosstalk includes electromagnetic interference (EMI) between adjacent conductors. Crosstalk may include inductive and/or capacitive coupling between conductors. Twisted pair wiring may reduce crosstalk by cross-cancelling EMI that would otherwise radiate out of the pair. Twisted pair wiring may cancel EMI by transmitting opposite signals in each of a twisted pair of conductors, such that the EMI from each individual wire is equal but opposite in polarity to the other wire. That is, the EMI from a first wire may be equal and opposite of the EMI from the second wire, and thus the two signals are substantially cancelled. For periodic signals, for example a sine wave, the signal in the second wire may be 180 degrees out-of-phase with the first wire.

Switching noise may occur in circuits when a switch changes from an on to off position. Switching noise may include transients, spikes, and other noise associated with switching. Switching noise in a mechanical switch may occur as a result of electrical contacts bouncing or chattering as they come into contact. Switching noise may be associated with analog mechanical switches, relays, capacitive discharge, momentary changes in ground levels, slew rates of digital and analog devices, any other suitable switching noise contributions, or any combination thereof.

It will be understood that, as used herein, signal amplitude refers to the peak deviation from zero. Thus a 1V sine wave has a 2V peak-to-peak amplitude. It will also be understood that phase refers to a fraction of a periodic cycle that has elapsed relative to an origin. Thus in two sine waves of the same frequency that are 180 degrees (or π radians) out-of-phase with one another, one wave is at a maximum when the other is at a minimum. This may also be referred to as an antiphase relationship.

The foregoing techniques may be implemented in an oximeter. An oximeter is a medical device that may determine the oxygen saturation of an analyzed tissue. One common type of oximeter is a pulse oximeter, which may non-invasively measure the oxygen saturation of a patient's blood (as opposed to measuring oxygen saturation directly by analyzing a blood sample taken from the patient). Pulse oximeters may be included in patient monitoring systems that measure and display various blood flow characteristics including, but not limited to, the oxygen saturation of hemoglobin in arterial blood. Such patient monitoring systems may also measure and display additional physiological parameters, such as a patient's pulse rate, respiration rate, respiration effort, blood pressure, any other suitable physiological parameter, or any combination thereof. Exemplary embodiments of determining respiration rate are disclosed in Addison et al. U.S. Patent Publication No. 2011/0071406, published Mar. 24, 2011, which is hereby incorporated by reference herein in its entirety. Exemplary embodiments of determining respiration effort are disclosed in Addison et al. U.S. Patent Publication No. 2011/0004081, published Jan. 6, 2011, which is hereby incorporated by reference herein in its entirety. Exemplary embodiments of determining blood pressure are disclosed in Addison et al. U.S. Patent Publication No. 2011/0028854, published Feb. 3, 2011, which is hereby incorporated by reference herein in its entirety. Pulse oximetry may be implemented using a photoplethysmograph. Pulse oximeters and other photoplethysmograph devices may also be used to determine other physiological parameter and information as disclosed in: J. Allen, “Photoplethysmography and its application in clinical physiological measurement,” Physiol. Meas., vol. 28, pp. R1-R39, March 2007; W. B. Murray and P. A. Foster, “The peripheral pulse wave: information overlooked,” J. Clin. Monit., vol. 12, pp. 365-377, September 1996; and K. H. Shelley, “Photoplethysmography: beyond the calculation of arterial oxygen saturation and heart rate,” Anesth. Analg., vol. 105, pp. S31-S36, December 2007; all of which are incorporated by reference herein in their entireties.

An oximeter may include a light sensor that is placed at a site on a patient, typically a fingertip, toe, forehead or earlobe, or in the case of a neonate, across a foot or hand. The oximeter may use a light source to pass light through blood perfused tissue and photoelectrically sense the absorption of the light in the tissue. In addition, locations which are not typically understood to be optimal for pulse oximetry serve as suitable sensor locations for the blood pressure monitoring processes described herein, including any location on the body that has a strong pulsatile arterial flow. For example, additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. Suitable sensors for these locations may include sensors for sensing absorbed light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some embodiments, the photonic signal interacting with the tissue is of one or more wavelengths that are attenuated by the blood in an amount representative of the blood constituent concentration. Red and infrared (IR) wavelengths may be used because it has been observed that highly oxygenated blood will absorb relatively less red light and more IR light than blood with a lower oxygen saturation. By comparing the intensities of two wavelengths at different points in the pulse cycle, it is possible to estimate the blood oxygen saturation of hemoglobin in arterial blood.

The system may process data to determine physiological parameters using techniques well known in the art. For example, the system may determine blood oxygen saturation using two wavelengths of light and a ratio-of-ratios calculation. The system also may identify pulses and determine pulse amplitude, respiration, blood pressure, other suitable parameters, or any combination thereof, using any suitable calculation techniques. In some embodiments, the system may use information from external sources (e.g., tabulated data, secondary sensor devices) to determine physiological parameters.

In some embodiments, a light drive modulation may be used. For example, a first light source may be turned on for a first drive pulse, followed by an off period, followed by a second light source for a second drive pulse, followed by an off period. The first and second drive pulses may be used to determine physiological parameters. The off periods may be used to detect ambient signal levels, reduce overlap of the light drive pulses, allow time for light sources to stabilize, allow time for detected light signals to stabilize or settle, reduce heating effects, reduce power consumption, for any other suitable reason, or any combination thereof.

It will be understood that the differential light drive techniques described herein are not limited to pulse oximeters and may be applied to any suitable medical and non-medical devices. For example, the system may use a differential light drive in a system for determining parameters such as regional saturation (rSO2), respiration rate, respiration effort, continuous non-invasive blood pressure, oxygen saturation pattern detection, fluid responsiveness, cardiac output, any other suitable clinical parameter, or any combination thereof.

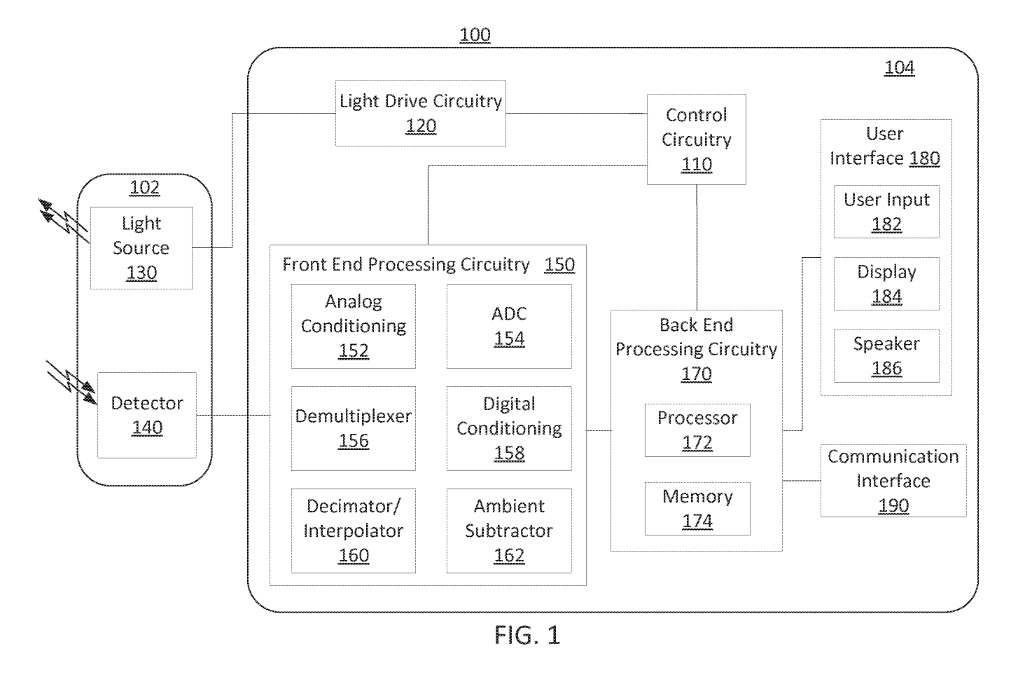

The following description and accompanying FIGS. 1-8 provide additional details and features of some embodiments of the present disclosure.

FIG. 1 is a block diagram of an illustrative physiological monitoring system 100 in accordance with some embodiments of the present disclosure. System 100 may include a sensor 102 and a monitor 104 for generating and processing physiological signals of a subject. In some embodiments, sensor 102 and monitor 104 may be part of an oximeter. In some embodiments, all or some of sensor 102, monitor 104, or both, may be referred to collectively as processing equipment.

Sensor 102 of physiological monitoring system 100 may include light source 130 and detector 140. Light source 130 may be configured to emit photonic signals having one or more wavelengths of light (e.g. red and IR) into a subject's tissue. For example, light source 130 may include a red light emitting light source and an IR light emitting light source, e.g. red and IR light emitting diodes (LEDs), for emitting light into the tissue of a subject to generate physiological signals. In one embodiment, the red wavelength may be between about 600 nm and about 750 nm, and the IR wavelength may be between about 800 nm and about 1000 nm. It will be understood that light source 130 may include any number of light sources with any suitable wavelengths and other characteristics. In embodiments where an array of sensors is used in place of single sensor 102, each sensor may be configured to emit a single wavelength. For example, a first sensor may emit only a red light while a second may emit only an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 140 may be chosen to be specifically sensitive to the chosen targeted energy spectrum of light source 130.

In some embodiments, detector 140 may be configured to detect the intensity of light at the red and IR wavelengths. In some embodiments, an array of sensors may be used and each sensor in the array may be configured to detect an intensity of a single wavelength. In operation, light may enter detector 140 after passing through the subject's tissue. Detector 140 may convert the intensity of the received light into an electrical signal. The light intensity may be directly related to the absorbance and/or reflectance of light in the tissue. That is, when more light at a certain wavelength is absorbed or reflected, less light of that wavelength is received from the tissue by detector 140. After converting the received light to an electrical signal, detector 140 may send the detection signal to monitor 104, where the detection signal may be processed and physiological parameters may be determined (e.g., based on the absorption of the red and IR wavelengths in the subject's tissue). In some embodiments, the detection signal may be preprocessed by sensor 102 before being transmitted to monitor 104.

In the embodiment shown, monitor 104 includes control circuitry 110, light drive circuitry 120, front end processing circuitry 150, back end processing circuitry 170, user interface 180, and communication interface 190. Monitor 104 may be communicatively coupled to sensor 102.

Control circuitry 110 may be coupled to light drive circuitry 120, front end processing circuitry 150, and back end processing circuitry 170, and may be configured to control the operation of these components. In some embodiments, control circuitry 110 may be configured to provide timing control signals to coordinate their operation. For example, light drive circuitry 120 may generate one or more light drive signals, which may be used to turn on and off the light source 130, based on the timing control signals. The front end processing circuitry 150 may use the timing control signals to operate synchronously with light drive circuitry 120. For example, front end processing circuitry 150 may synchronize the operation of an analog-to-digital converter and a demultiplexer with the light drive signal based on the timing control signals. In addition, the back end processing circuitry 170 may use the timing control signals to coordinate its operation with front end processing circuitry 150.

Light drive circuitry 120, as discussed above, may be configured to generate a light drive signal that is provided to light source 130 of sensor 102. The light drive signal may, for example, control the intensity of light source 130 and the timing of when light source 130 is turned on and off. In some embodiments, light drive circuitry 130 provides one or more differential light drive signals to light source 130. Where light source 130 is configured to emit two or more wavelengths of light, the light drive signal may be configured to control the operation of each wavelength of light. The light drive signal may comprise a single signal or may comprise multiple signals (e.g., one signal for each wavelength of light).

In some embodiments, control circuitry 110 and light drive circuitry 120 may generate light drive parameters based on a metric. For example, back end processing 170 may receive information about received light signals, determine light drive parameters based on that information, and send corresponding information to control circuitry 110.

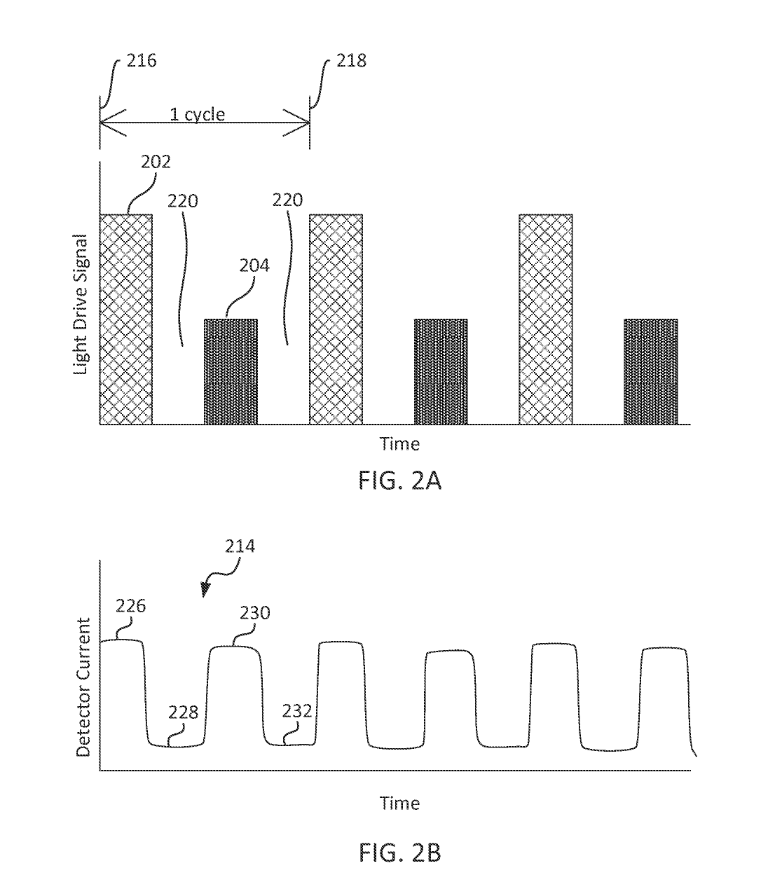

FIG. 2A shows an illustrative plot of a light drive signal including red light drive pulse 202 and IR light drive pulse 204 in accordance with some embodiments of the present disclosure. In some embodiments, FIG. 2A represents an idealized multiplexing of emitted light signals, where the pulses correspond to the system turning light signals on and off using a differential light drive. It will be understood that light drive signals may include square waves, sine waves, shaped pulses, any other suitable light drive signal, or any combination thereof. The timing and shape of the pulses may be controlled using one or more unipolar power supplies, digital signal generators, digital filters, analog filters, any other suitable equipment, or any combination thereof. For example, light drive pulses 202 and 204 may be generated by light drive circuitry 120 under the control of control circuitry 110. As used herein, drive pulses may refer to the high and low states of a pulse, switching power or other components on and off, high and low output states, high and low values within a continuous modulation, other suitable relatively distinct states, or any combination thereof. The light drive signal may be provided to light source 130, including red light drive pulse 202 and IR light drive pulse 204 to drive red and IR light emitters, respectively, within light source 130.

Red light drive pulse 202 may have a higher amplitude than IR light drive 204 since red LEDs may be less efficient than IR LEDs at converting electrical energy into light energy. In some embodiments, the output levels may be equal, may be adjusted for nonlinearity of emitters, may be modulated in any other suitable technique, or any combination thereof. Additionally, red light may be absorbed and scattered more than IR light when passing through perfused tissue.

When the red and IR light sources are driven in this manner they emit pulses of light at their respective wavelengths into the tissue of a subject in order generate physiological signals that physiological monitoring system 100 may process to calculate physiological parameters. It will be understood that the light drive amplitudes of FIG. 2A are merely exemplary and that any suitable amplitudes or combination of amplitudes may be used, and may be based on the light sources, the subject tissue, the determined physiological parameter, modulation techniques, power sources, any other suitable criteria, or any combination thereof.

The light drive signal of FIG. 2A may also include “off” periods 220 between the red and IR light drive pulse. “Off” periods 220 are periods during which no drive current may be applied to light source 130. “Off” periods 220 may be provided, for example, to prevent overlap of the emitted light, since light source 130 may require time to turn completely on and completely off. The period from time 216 to time 218 may be referred to as a drive cycle, which includes four segments: a red light drive pulse 202, followed by an “off” period 220, followed by an IR light drive pulse 204, and followed by an “off” period 220. After time 218, the drive cycle may be repeated (e.g., as long as a light drive signal is provided to light source 130). It will be understood that the starting point of the drive cycle is merely illustrative and that the drive cycle can start at any location within FIG. 2A, provided the cycle spans two drive pulses and two “off” periods. Thus, each red light drive pulse 202 and each IR light drive pulse 204 may be understood to be surrounded by two “off” periods 220. “Off” periods may also be referred to as dark periods, in that the emitters are dark or returning to dark during that period. It will be understood that the particular square pulses illustrated in FIG. 2A are merely exemplary and that any suitable light drive scheme is possible. For example, light drive schemes may include shaped pulses, sinusoidal modulations, time division multiplexing other than as shown, frequency division multiplexing, phase division multiplexing, any other suitable light drive scheme, or any combination thereof.

Referring back to FIG. 1, front end processing circuitry 150 may receive a detection signal from detector 140 and provide one or more processed signals to back end processing circuitry 170. The term “detection signal,” as used herein, may refer to any of the signals generated within front end processing circuitry 150 as it processes the output signal of detector 140. Front end processing circuitry 150 may perform various analog and digital processing of the detector signal. One suitable detector signal that may be received by front end processing circuitry 150 is shown in FIG. 2B.

FIG. 2B shows an illustrative plot of detector current waveform 214 that may be generated by a sensor in accordance with some embodiments of the present disclosure. The peaks of detector current waveform 214 may represent current signals provided by a detector, such as detector 140 of FIG. 1, when light is being emitted from a light source. The amplitude of detector current waveform 214 may be proportional to the light incident upon the detector. The peaks of detector current waveform 214 may be synchronous with drive pulses driving one or more emitters of a light source, such as light source 130 of FIG. 1. For example, detector current peak 226 may be generated in response to a light source being driven by red light drive pulse 202 of FIG. 2A, and peak 230 may be generated in response to a light source being driven by IR light drive pulse 204. Valley 228 of detector current waveform 214 may be synchronous with periods of time during which no light is being emitted by the light source, or the light source is returning to dark, such as “off” period 220. While no light is being emitted by a light source during the valleys, detector current waveform 214 may not fall all of the way to zero.

It will be understood that detector current waveform 214 may be an at least partially idealized representation of a detector signal, assuming perfect light signal generation, transmission, and detection. It will be understood that an actual detector current will include amplitude fluctuations, frequency deviations, droop, overshoot, undershoot, rise time deviations, fall time deviations, other deviations from the ideal, or any combination thereof. It will be understood that the system may shape the drive pulses shown in FIG. 2A in order to make the detector current as similar as possible to idealized detector current waveform 214.

Referring back to FIG. 1, front end processing circuitry 150, which may receive a detection signal, such as detector current waveform 214, may include analog conditioning 152, analog-to-digital converter (ADC) 154, demultiplexer 156, digital conditioning 158, decimator/interpolator 160, and ambient subtractor 162.

Analog conditioning 152 may perform any suitable analog conditioning of the detector signal. The conditioning performed may include any type of filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof.

The conditioned analog signal may be processed by analog-to-digital converter 154, which may convert the conditioned analog signal into a digital signal. Analog-to-digital converter 154 may operate under the control of control circuitry 110. Analog-to-digital converter 154 may use timing control signals from control circuitry 110 to determine when to sample the analog signal. Analog-to-digital converter 154 may be any suitable type of analog-to-digital converter of sufficient resolution to enable a physiological monitor to accurately determine physiological parameters.

Demultiplexer 156 may operate on the analog or digital form of the detector signal to separate out different components of the signal. For example, detector current waveform 214 of FIG. 2B includes a red component corresponding to peak 226, an IR component corresponding to peak 230, and at least one ambient component corresponding to valley 230. Demultiplexer 156 may operate on detector current waveform 214 of FIG. 2B to generate a red signal, an IR signal, a first ambient signal (e.g., corresponding to the ambient component corresponding to valley 230 that occurs immediately after the peak 226), and a second ambient signal (e.g., corresponding to the ambient component corresponding to valley 230 that occurs immediately after the IR component 230). Demultiplexer 156 may operate under the control of control circuitry 110. For example, demultiplexer 156 may use timing control signals from control circuitry 110 to identify and separate out the different components of the detector signal.

Digital conditioning 158 may perform any suitable digital conditioning of the detector signal. Digital conditioning 158 may include any type of digital filtering of the signal (e.g., low pass, high pass, band pass, notch, or any other suitable filtering), amplifying, performing an operation on the signal, performing any other suitable digital conditioning, or any combination thereof.

Decimator/interpolator 160 may decrease the number of samples in the digital detector signal. For example, decimator/interpolator 160 may decrease the number of samples by removing samples from the detector signal or replacing samples with a smaller number of samples. The decimation or interpolation operation may include or be followed by filtering to smooth the output signal.

Ambient subtractor 162 may operate on the digital signal. In some embodiments, ambient subtractor 162 may remove dark or ambient contributions to the received signal.

The components of front end processing circuitry 150 are merely illustrative and any suitable components and combinations of components may be used to perform the front end processing operations.

The front end processing circuitry 150 may be configured to take advantage of the full dynamic range of analog-to-digital converter 154. This may be achieved by applying gain to the detection signal by analog conditioning 152 to map the expected range of the detection signal to the full or close to full output range of analog-to-digital converter 154. The output value of analog-to-digital converter 154, as a function of the total analog gain applied to the detection signal, may be given as:

ADC Value=Total Analog Gain×[Ambient Light+LED Light]

Ideally, when ambient light is zero and when the light source is off, the analog-to-digital converter 154 will read just above the minimum input value. When the light source is on, the total analog gain may be set such that the output of analog-to-digital converter 154 may read close to the full scale of analog-to-digital converter 154 without saturating. This may allow the full dynamic range of analog-to-digital converter 154 to be used for representing the detection signal, thereby increasing the resolution of the converted signal. In some embodiments, the total analog gain may be reduced by a small amount so that small changes in the light level incident on the detector do not cause saturation of analog-to-digital converter 154.

However, if the contribution of ambient light is large relative to the contribution of light from a light source, the total analog gain applied to the detection current may need to be reduced to avoid saturating analog-to-digital converter 154. When the analog gain is reduced, the portion of the signal corresponding to the light source may map to a smaller number of analog-to-digital conversion bits. Thus, more ambient light noise in the input of analog-to-digital converter 154 may results in fewer bits of resolution for the portion of the signal from the light source. This may have a detrimental effect on the signal-to-noise ratio of the detection signal. Accordingly, passive or active filtering or signal modification techniques may be employed to reduce the effect of ambient light on the detection signal that is applied to analog-to-digital converter 154, and thereby reduce the contribution of the noise component to the converted digital signal.

Back end processing circuitry 170 may include processor 172 and memory 174. Processor 172 may be adapted to execute software, which may include an operating system and one or more applications, as part of performing the functions described herein. Processor 172 may receive and further physiological signals received from front end processing circuitry 150. For example, processor 172 may determine one or more physiological parameters based on the received physiological signals. Processor 172 may include an assembly of analog or digital electronic components. Processor 172 may calculate physiological information. For example, processor 172 may compute one or more of a pulse rate, respiration rate, blood pressure, or any other suitable physiological parameter. Processor 172 may perform any suitable signal processing of a signal, such as any suitable band-pass filtering, adaptive filtering, closed-loop filtering, any other suitable filtering, and/or any combination thereof. Processor 172 may also receive input signals from additional sources not shown. For example, processor 172 may receive an input signal containing information about treatments provided to the subject from user interface 180. Additional input signals may be used by processor 172 in any of the calculations or operations it performs in accordance with back end processing circuitry 170 or monitor 104.

Memory 174 may include any suitable computer-readable media capable of storing information that can be interpreted by processor 172. In some embodiments, memory 174 may store calculated values, such as pulse rate, blood pressure, blood oxygen saturation, fiducial point locations or characteristics, initialization parameters, any other calculated values, or any combination thereof, in a memory device for later retrieval. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system. Back end processing circuitry 170 may be communicatively coupled with user interface 180 and communication interface 190.

User interface 180 may include user input 182, display 184, and speaker 186. User interface 180 may include, for example, any suitable device such as one or more medical devices (e.g., a medical monitor that displays various physiological parameters, a medical alarm, or any other suitable medical device that either displays physiological parameters or uses the output of back end processing 170 as an input), one or more display devices (e.g., monitor, personal digital assistant (PDA), mobile phone, tablet computer, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

User input 182 may include any type of user input device such as a keyboard, a mouse, a touch screen, buttons, switches, a microphone, a joy stick, a touch pad, or any other suitable input device. The inputs received by user input 182 can include information about the subject, such as age, weight, height, diagnosis, medications, treatments, and so forth.

In an embodiment, the subject may be a medical patient and display 184 may exhibit a list of values which may generally apply to the patient, such as, for example, age ranges or medication families, which the user may select using user input 182. Additionally, display 184 may display, for example, an estimate of a subject's blood oxygen saturation generated by monitor 104 (referred to as an “SpO₂” measurement), pulse rate information, respiration rate information, blood pressure, any other parameters, and any combination thereof. Display 184 may include any type of display such as a cathode ray tube display, a flat panel display such a liquid crystal display or plasma display, or any other suitable display device. Speaker 186 within user interface 180 may provide an audible sound that may be used in various embodiments, such as for example, sounding an audible alarm in the event that a patient's physiological parameters are not within a predefined normal range.

Communication interface 190 may enable monitor 104 to exchange information with external devices. Communications interface 190 may include any suitable hardware, software, or both, which may allow monitor 104 to communicate with electronic circuitry, a device, a network, a server or other workstations, a display, or any combination thereof. Communications interface 190 may include one or more receivers, transmitters, transceivers, antennas, plug-in connectors, ports, communications buses, communications protocols, device identification protocols, any other suitable hardware or software, or any combination thereof. Communications interface 190 may be configured to allow wired communication (e.g., using USB, RS-232, Ethernet, or other standards), wireless communication (e.g., using WiFi, IR, WiMax, BLUETOOTH, USB, or other standards), or both. For example, communications interface 190 may be configured using a universal serial bus (USB) protocol (e.g., USB 2.0, USB 3.0), and may be configured to couple to other devices (e.g., remote memory devices storing templates) using a four-pin USB standard Type-A connector (e.g., plug and/or socket) and cable. In some embodiments, communications interface 190 may include an internal bus such as, for example, one or more slots for insertion of expansion cards.

It will be understood that the components of physiological monitoring system 100 that are shown and described as separate components are shown and described as such for illustrative purposes only. In some embodiments the functionality of some of the components may be combined in a single component. For example, the functionality of front end processing circuitry 150 and back end processing circuitry 170 may be combined in a single processor system. Additionally, in some embodiments the functionality of some of the components of monitor 104 shown and described herein may be divided over multiple components. For example, some or all of the functionality of control circuitry 110 may be performed in front end processing circuitry 150, in back end processing circuitry 170, or both. In other embodiments, the functionality of one or more of the components may be performed in a different order or may not be required. In an embodiment, all of the components of physiological monitoring system 100 can be realized in processor circuitry.

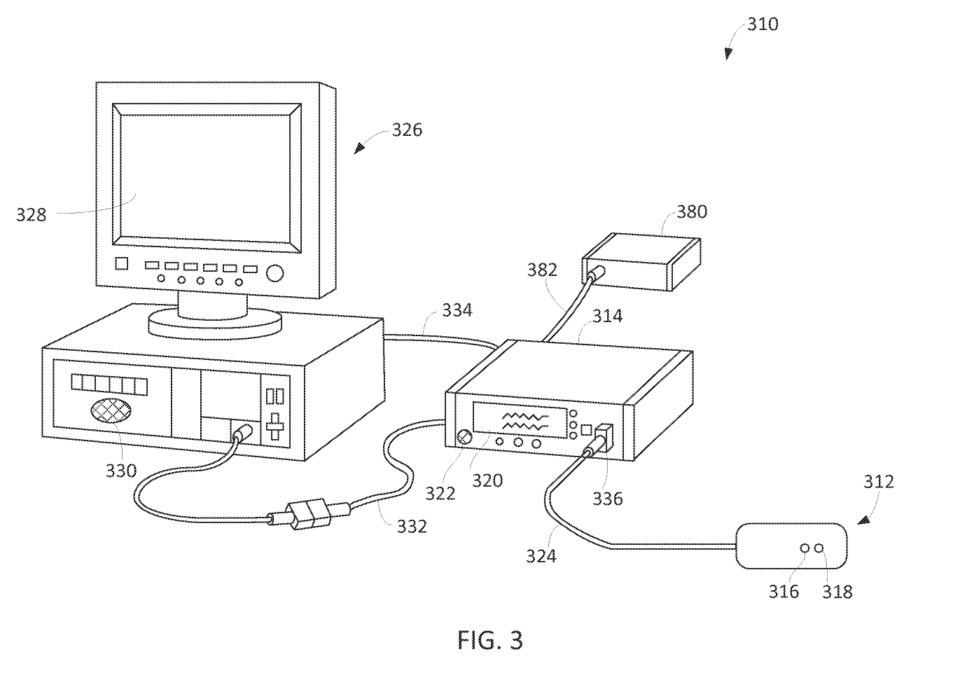

FIG. 3 is a perspective view of an embodiment of a physiological monitoring system 310 in accordance with some embodiments of the present disclosure. In some embodiments, one or more components of physiological monitoring system 310 may include one or more components of physiological monitoring system 100 of FIG. 1. Physiological monitoring system 310 may include sensor unit 312 and monitor 314. In some embodiments, sensor unit 312 may be part of an oximeter. Sensor unit 312 may include one or more light source 316 for emitting light at one or more wavelengths into a subject's tissue. One or more detector 318 may also be provided in sensor unit 312 for detecting the light that is reflected by or has traveled through the subject's tissue. Any suitable configuration of light source 316 and detector 318 may be used. In an embodiment, sensor unit 312 may include multiple light sources and detectors, which may be spaced apart. Physiological monitoring system 310 may also include one or more additional sensor units (not shown) that may, for example, take the form of any of the embodiments described herein with reference to sensor unit 312. An additional sensor unit may be the same type of sensor unit as sensor unit 312, or a different sensor unit type than sensor unit 312 (e.g., a photoacoustic sensor). Multiple sensor units may be capable of being positioned at two different locations on a subject's body.

In some embodiments, sensor unit 312 may be connected to monitor 314 as shown. Sensor unit 312 may be powered by an internal power source, e.g., a battery (not shown). Sensor unit 312 may draw power from monitor 314. In another embodiment, the sensor may be wirelessly connected (not shown) to monitor 314. Monitor 314 may be configured to calculate physiological parameters based at least in part on data relating to light emission and acoustic detection received from one or more sensor units such as sensor unit 312. For example, monitor 314 may be configured to determine pulse rate, respiration rate, respiration effort, blood pressure, blood oxygen saturation (e.g., arterial, venous, or both), hemoglobin concentration (e.g., oxygenated, deoxygenated, and/or total), any other suitable physiological parameters, or any combination thereof. In some embodiments, calculations may be performed on the sensor units or an intermediate device and the result of the calculations may be passed to monitor 314. Further, monitor 314 may include display 320 configured to display the physiological parameters or other information about the system. In the embodiment shown, monitor 314 may also include a speaker 322 to provide an audible sound that may be used in various other embodiments, such as for example, sounding an audible alarm in the event that a subject's physiological parameters are not within a predefined normal range. In some embodiments, physiological monitoring system 310 may include a stand-alone monitor in communication with the monitor 314 via a cable or a wireless network link. In some embodiments, monitor 314 may be implemented as display 184 of FIG. 1.

In some embodiments, sensor unit 312 may be communicatively coupled to monitor 314 via a cable 324 at port 336. Cable 324 may include electronic conductors (e.g., wires for transmitting electronic signals from detector 318), optical fibers (e.g., multi-mode or single-mode fibers for transmitting emitted light from light source 316), any other suitable components, any suitable insulation or sheathing, or any combination thereof. In some embodiments, a wireless transmission device (not shown) or the like may be used instead of or in addition to cable 324. Monitor 314 may include a sensor interface configured to receive physiological signals from sensor unit 312, provide signals and power to sensor unit 312, or otherwise communicate with sensor unit 312. The sensor interface may include any suitable hardware, software, or both, which may be allow communication between monitor 314 and sensor unit 312.

In some embodiments, physiological monitoring system 310 may include calibration device 380. Calibration device 380, which may be powered by monitor 314, a battery, or by a conventional power source such as a wall outlet, may include any suitable calibration device. Calibration device 380 may be communicatively coupled to monitor 314 via communicative coupling 382, and/or may communicate wirelessly (not shown). In some embodiments, calibration device 380 is completely integrated within monitor 314. In some embodiments, calibration device 380 may include a manual input device (not shown) used by an operator to manually input reference signal measurements obtained from some other source (e.g., an external invasive or non-invasive physiological measurement system).

In the illustrated embodiment, physiological monitoring system 310 includes a multi-parameter physiological monitor 326. The monitor 326 may include a cathode ray tube display, a flat panel display (as shown) such as a liquid crystal display (LCD) or a plasma display, or may include any other type of monitor now known or later developed. Multi-parameter physiological monitor 326 may be configured to calculate physiological parameters and to provide a display 328 for information from monitor 314 and from other medical monitoring devices or systems (not shown). For example, multi-parameter physiological monitor 326 may be configured to display an estimate of a subject's blood oxygen saturation and hemoglobin concentration generated by monitor 314. Multi-parameter physiological monitor 326 may include a speaker 330.

Monitor 314 may be communicatively coupled to multi-parameter physiological monitor 326 via a cable 332 or 334 that is coupled to a sensor input port or a digital communications port, respectively and/or may communicate wirelessly (not shown). In addition, monitor 314 and/or multi-parameter physiological monitor 326 may be coupled to a network to enable the sharing of information with servers or other workstations (not shown). Monitor 314 may be powered by a battery (not shown) or by a conventional power source such as a wall outlet.

In some embodiments, all or some of monitor 314 and multi-parameter physiological monitor 326 may be referred to collectively as processing equipment.

In some embodiments, any of the processing components and/or circuits, or portions thereof, of FIGS. 1 and 3 may be referred to collectively as processing equipment. For example, processing equipment may be configured to amplify, filter, sample and digitize an input signal from sensor 102 (e.g., using an analog-to-digital converter), and calculate physiological information from the digitized signal. Processing equipment may be configured to generate one or more differential light drive signals, control one or more voltage and/or current sources, amplify, filter, sample and digitize detector signals, and calculate physiological information from the digitized signal. In some embodiments, all or some of the components of the processing equipment may be referred to as a processing module.

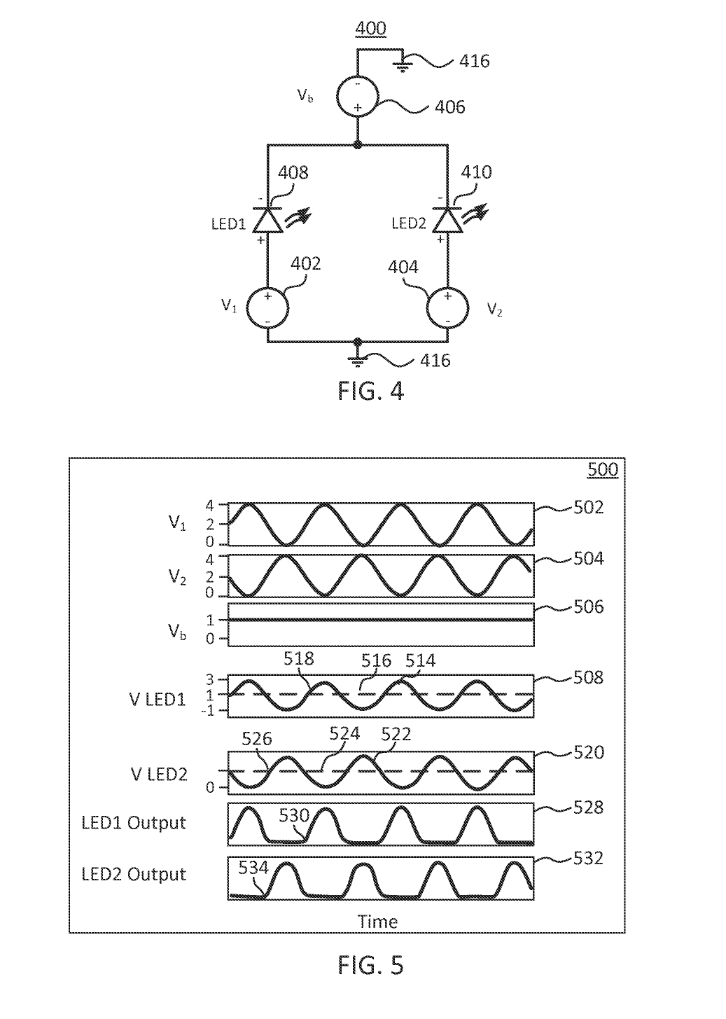

FIG. 4 shows illustrative circuit 400 including two AC voltage sources in a differential light drive in accordance with some embodiments of the present disclosure. Circuit 400 includes first voltage source V₁ 402, second voltage source V₂ 404, and third voltage source V_{b} 406. Circuit 400 includes ground 416, LED1 408 and LED2 410. The positive and negative symbols indicate the anode and cathode of the LEDs, respectively. As used herein, a voltage of 5V across an LED indicates that the anode (“+” side) is at a voltage 5V more than the cathode (“−” side). In some embodiments, a differential light drive may be provided to LED1 408 and LED2 410 by modulating V₁ 402 and V₂ 404. The illumination of LED1 408 and LED2 410 is determined based on the differential voltage across the components.

FIG. 5 shows illustrative signal plot window 500 corresponding to using two AC voltage sources in a differential light drive in accordance with some embodiments of the present disclosure. In some embodiments, plot window 500 includes signals associated with circuit 400 of FIG. 4. Window 500 includes V₁ plot 502, V₂ plot 504, V_{b} plot 506, V LED1 plot 508, V LED2 plot 520, LED1 Output plot 528, and LED2 Output plot 532. The plots of plot window 500 share a common x-axis of time. The y-axes of the plots in plot window 500 correspond to signal amplitude and need not be drawn to scale. Amplitude may include voltage, current, power, brightness, any other suitable metric, or any combination thereof. It will be understood that the particular plots shown, and the signals of those plots, are merely exemplary. For example, plot 502 and 504 may include square waves, triangle waves, saw-tooth waves, more complex waveforms, any other suitable signal, or any combination thereof. It will also be understood that signals may corresponds to voltage signals, current signals, power signals, light intensity signals, any other suitable amplitude units, or any combination thereof.

Plot 502 corresponds to the voltage output of voltage source V₁402 of FIG. 4. Plot 504 corresponds to the voltage output of voltage source V₂ 404 of FIG. 4. Plot 506 corresponds to the voltage output of V_{b} 406 of FIG. 4. In some embodiments, V_{b} corresponds to a bias voltage. In the illustrated embodiment, V_{b} is a DC voltage signal, though it will be understood that V_{b} need not be a constant signal. It will also be understood that in some embodiments, current sources may be used in addition to or in place of the illustrated voltage sources.

Referring back to FIG. 4, the voltage across LED1 408 corresponds to the difference in voltage at the negative terminal of V_{b} 406 and the positive terminal of V₁ 402. The voltage across LED2 410 corresponds to the difference in voltage at the negative terminal of V_{b} 406 and the positive terminal of V₂ 404.

In FIG. 5, plot 508 shows the voltage across LED1 408 of FIG. 4 and plot 520 shows the voltage across LED2 410 of FIG. 4. Referring back to FIG. 4, 1V is applied to the cathode of LED1 408 by V_{b} 406, and a 2V sine wave with a 2V offset is applied to the anode of LED1 408. It will be understood that, as used herein, amplitude refers to the peak deviation from zero. Thus a 2V sine wave has a 4V peak-to-peak amplitude. As shown in plot 508, the voltage across LED1 is a 2V sine wave that varies from −1V to 3V in absolute amplitude in phase with the voltage V₁ shown in plot 502. For example, when the voltage at V₁, and thus the voltage of the anode of the LED is 4V, and the voltage from V_{b}, and thus the voltage at the cathode of the LED is 1V, the differential voltage across the LED is 3V (where 4−1=3).

Plot 520 shows the voltage across LED2 410 of FIG. 4. The signal in plot 520 shows similar behavior to that of the voltage across LED1 408 of FIG. 4, except that the voltage across LED2 410 of FIG. 4 is in phase with the voltage signal from V₂ 404 of FIG. 4. In the illustrated example, V₁ and V₂ are both 2V sine waves, and they are 180 degrees out-of-phase with one another.

Plot 528 corresponds to the illumination of LED1 408 of FIG. 4. Plot 532 corresponds to the illumination of LED2 410 of FIG. 4. For example, the signal amplitude in plots 528 and 532 may correspond to current through the LED, lumens emitted by the LED, any other suitable units, or any combination thereof.

In plot 508, voltage signal 514 is indicated as a solid line and threshold 516 is indicated as a dashed line. In the illustrated embodiment, threshold 516 corresponds to the turn-on voltage of LED1. As illustrated, the turn-on voltage is approximately 1V, and thus, light is emitted from the LED when the voltage is at or above 1V. At time point 518, voltage signal 514 exceeds threshold 516. Concurrently, at time point 530 shown in plot 528, the light output from LED1 changes from an off state to an on state.

In plot 520, voltage signal 522 is indicated as a solid line and threshold 524 is indicated as a dashed line. In the illustrated embodiment, threshold 524 corresponds to the turn-on voltage of LED2. At time point 526, voltage signal 522 exceeds threshold 524. Concurrently, at time point 534 shown in plot 532, the light output from LED2 changes from an off state to an on state.

Plots 528 and 532 show that the output from LED 1 and LED2 are controlled by the voltage levels V₁ and V₂ in plots 502 and 504. The illumination of LED1 and LED2 are 180 degrees out-of-phase, and correspond to the phase difference between V₁ and V₂. In some embodiments, the out-of-phase signals V₁ and V₂ will reduce interference and noise. For example, the signals may be carried in a twisted pair of wires, such that EMI associated with the signals is cancelled.

It will be understood that the particular signals shown in window 500 of FIG. 5 that correspond to elements of circuit 400 of FIG. 4 are merely exemplary, and that any suitable signals may be used. For example, signals need not be 180 degrees out-of-phase, and thus can be used to turn any suitable number of LEDs on and off at any suitable times. In another example, periodic signals other than sine waves may be used, such as square waves, triangle waves, pulse-width modulated waves, waves of any suitable shape, or any combination thereof. It will also be understood that signals need not be periodic. For example, voltage and/or current sources may be controlled by a DAC and may assume any suitable levels. It will also be understood that circuit 400 may be used with any suitable number of voltage sources and any suitable number of emitters. In some embodiments, the bias voltage source V_{b} may be omitted or replaced.

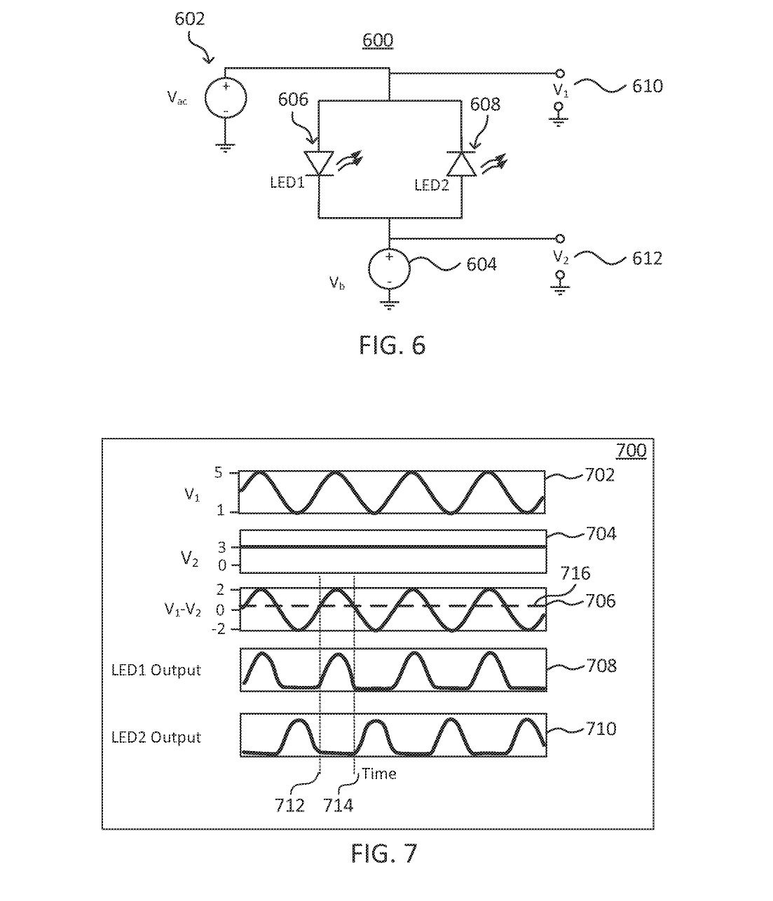

FIG. 6 shows illustrative circuit 600 including one AC voltage source in a differential light drive in accordance with some embodiments of the present disclosure. Circuit 600 includes AC voltage source V_{ac} 602, bias voltage source V_{b} 604, LED1 606, and LED2 608. Voltage V₁ 610 corresponds to the voltage at the anode of LED1 606 and the cathode of LED2 608. V₁ also corresponds to the positive voltage output of V_{ac} 602. Voltage V₂ 612 corresponds to the voltage at the cathode of LED1 606 and the anode of LED2 608. V₂ 612 also corresponds to the positive voltage output of V_{b} 604.

LED1 606 and LED2 608 are wired in a back-to-back or opposite-parallel configuration. Thus, when V₁ 610 is greater than V₂ 612, current flows through LED1 606. Conversely, when V₁ 610 is less than V₂ 612, current flows through LED2 608. The signal difference between V₁ 610 and V₂ 612 corresponds to a differential light drive voltage. When the differential light drive voltage is above the turn-on voltage of one of the LEDs and an appropriate current flows through the device, light is emitted.

FIG. 7 shows illustrative signal plot window 700 corresponding to using one AC voltage source in a differential light drive in accordance with some embodiments of the present disclosure. In some embodiments, the plots of window 700 correspond to the elements of circuit 600 of FIG. 6. Plot 702 shows the amplitude of voltage V₁ 610 of FIG. 6. Plot 704 shows the amplitude of voltage V₂ 612 of FIG. 6. Plot 706 shows the voltage difference between V₁ and V₂. Plot 708 shows the light output of LED1 606 of FIG. 6. Plot 710 shows the light output of LED2 608 of FIG. 6. The plots of plot window 700 share a common x-axis of time. The y-axes of the plots in plot window 700 correspond to signal amplitude and need not be drawn to scale. It will be understood that the particular plots shown are merely exemplary. For example, plot 702 and 704 may include square waves, triangle waves, saw-tooth waves, more complex waveforms, any other suitable signal, or any combination thereof. It will also be understood that signals may corresponds to voltage signals, current signals, power signals, light intensity signals, any other suitable amplitude units, or any combination thereof.

In the illustrated example, a 2V sine wave with a 3V offset is provided by V_{ac} 602 of FIG. 6. Thus, plot 702 shows a sine wave with a maximum voltage of 5V and a minimum voltage of 1V. V_{b} 604 of FIG. 6 provides a 3V DC signal, as shown in plot 704. The differential voltage, that is, the voltage across the LEDs of circuit 600 of FIG. 6, is a 2V sine wave centered at 0V, as shown in plot 706. In the illustrated embodiment, this differential voltage that varies above and below 0V is provided using only positive power supply rails. It will be understood that in some embodiments, the signal offset need not be provided and that bipolar power supplies may be used.

When V₁-V₂ is positive, current flows through LED1 606 of FIG. 6. Assuming that the turn-on threshold voltage of the LED is exceed, light is emitted from LED1 606 of FIG. 6. The corresponding light output is shown in plot 708. Threshold 716 corresponds to the turn-on voltage of LED1 606 of FIG. 6. At time point 712, the signal in plot 706 exceeds threshold 716 and light begins to be emitted from LED1 606 of FIG. 6, as shown in plot 708. At time point 714, the signal in plot 706 falls below threshold 716, and light is no longer emitted from LED1 606 of FIG. 6 as shown in plot 708. Due to the back-to-back configuration of the LEDs in circuit 600 of FIG. 6, current does not flow through LED2 608 of FIG. 6 when it flows through LED1 606 of FIG. 6, and flows through LED2 608 of FIG. 6 when it does not flow through LED1 606 of FIG. 6. Thus, plot 710 shows that no light is emitted from LED2 between time points 712 and 714.

In some embodiments, such as the one illustrated in FIGS. 6 and 7, the current in the wire connecting one of the two voltage sources to the LEDs and the current in the wire connecting the other of the two voltage sources to the LEDs may be equal and 180 degrees out-of-phase. Thus, the two wires may be arranged as a twisted pair, or any other suitable technique, in order to reduce crosstalk and other noise. Switching noise may be avoided because this configuration allows the LEDs to be turned on and off without using switches. In some embodiments, only the far end crosstalk from one diode arrives at the other diode, and it arrives antiphase (i.e., 180 degrees out-of-phase) such that it helps turn off the victim diode (i.e., the diode at which the crosstalk arrives), which may be a beneficial effect.

It will be understood that the circuits illustrated in FIGS. 4 and 6 may include any suitable elements and may generate any suitable signals. For example, the circuits may include any suitable resistors, capacitors, inductors, amplifiers, current limiters, diodes, integrated circuit elements, processing equipment, any other suitable components, or any combination thereof. The circuits may include any suitable wiring and shielding techniques, including integrated circuits, twisted pair wiring, printed circuit boards, metallic shielding elements, any other suitable components, or any combination thereof. In some embodiments, the circuits may be implemented to provide a differential light drive signal using current sources, voltage sources, or a combination of current and voltage sources. Current and/or voltage sources may be controlled by a processor, for example using a digital-to-analog converter output, a computer-controlled current source, a computer-controlled voltage source, any other suitable technique, or any combination thereof. The generated signals may include any suitable signals including periodic sine waves, square waves, triangle waves, sawtooth waves, constant voltage signals, constant current signals, other shaped waves, any other suitable shape, or any combination thereof. It will also be understood that the signals need not be 180 degrees out-of-phase, and may include any suitable phase shifts in order to generate a desired light drive. In some embodiments, more than two LEDs may be driven using a differential light drive technique, where any suitable phase shifts are included to cancel crosstalk and/or other noise. In some embodiments, differential signals may include periods where no, one, or multiple LEDs are illuminated concurrently. For example, differential drive signals may include a period where no LEDs are illuminated in order to determine a dark signal level.

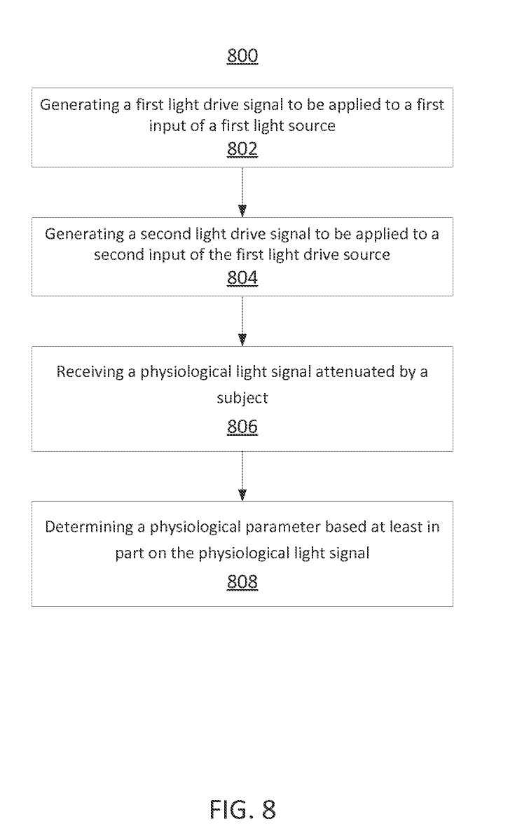

FIG. 8 shows an illustrative flow diagram including steps for using a differential light drive in accordance with some embodiments of the present disclosure.

Step 802 includes generating a first light drive signal to be applied to a first input of a first light source. For example, a light drive signal from V₁ 402 of FIG. 4 may be the first light drive signal and the first light source may be LED1 408 of FIG. 4. The first input of the first light drive source may be the anode of LED1 408 of FIG. 4. In another example, a light drive signal from V_{ac} 602 of FIG. 6 may be the first light drive signal, and the first light source may be LED1 606 of FIG. 6. The first light drive signal may include any suitable light drive signal as discussed above, for example, a sine wave.

Step 804 includes generating a second light drive signal to be applied to a second input of the first light drive source. For example, a light drive signal from V_{b} 406 may be the second light drive signal. The second input of the first light drive source may be the cathode of LED1 408 of FIG. 4. In another example, a light drive signal from V_{b} 604 of FIG. 6 may be the second drive signal. In some embodiments, the second light drive signal may include a DC bias voltage signal.

An optional step, not shown, includes generating a third light drive signal to be applied to a first input of a second light source, where the second light drive signal is further configured to be applied to a second input of the second light source, and where the third and second light drive signals together provide a second differential light drive signal to the second light source. For example, a light drive signal from V₂ 404 of FIG. 4 (i.e., the second light drive signal) may be a third light drive signal, which may combine with the V_{b} 406 of FIG. 4 to provide a differential light signal to second light emitter LED2 410 of FIG. 4.

Another optional step, not shown, includes connecting a second light source in a back-to-back configuration with the first light source, where the first and second light sources are LEDs. The differential light drive generated by the first and second light drive signals may alternately illuminate the first and second light sources, as illustrated in plot window 700 of FIG. 7.

Step 806 includes receiving a physiological light signal attenuated by a subject. In some embodiments, light signals generated by light emitters may be partially attenuated by a subject before being detected by the system. The light signals may be detected using any suitable photodetector. For example, a photodetector may include a solid state device that generates a current in response to absorbing light. It will be understood that any suitable photodetector or combination of photodetectors may be used to detect the attenuated light signal. The amount of attenuation may correspond to, in the example of a pulse oximeter, a volume of blood or other tissue through which the light has travelled.

Step 808 includes determining a physiological parameter based at least in part on the physiological light signal. Physiological parameters may include oxygen saturation, pulse rate, respiration rate, respiration effort, blood pressure, hemoglobin concentration, any other suitable parameter, or any combination thereof. In some embodiments, a photoplethysmographic signal may be generated based on the received physiological light signal, and may be used to determine physiological parameters. Determining physiological parameters may include applying filters, transforming, performing ratio-of-ratio calculations, peak finding, demultiplexing, amplifying, performing any other suitable techniques, or any combination thereof. In some embodiments, determined physiological parameters may be provided to a user and/or to equipment for further processing. For example, parameters may be displayed on a display screen, transmitted to another device for display, and/or transmitted to another device for further processing.

It will be understood that the steps above are exemplary and that in some implementations, steps may be added, removed, omitted, repeated, reordered, modified in any other suitable way, or any combination thereof. For example, steps 806 and 808 may be omitted.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed:

1. A system for driving a light source for a physiological monitor, the system comprising:
a first light drive signal generator configured to generate a first light drive signal to be applied to a first input of a first light source; and
a second light drive signal generator configured to generate a second light drive signal to be applied to a second input of the first light source, wherein the first light drive signal and the second light drive signals together provide a first differential light drive signal to the first light source.

2. The system of claim 1:
further comprising a third light drive signal generator configured to generate a third light drive signal to be applied to a first input of a second light source;
wherein the second light drive signal is further configured to be applied to a second input of the second light source; and
wherein the third and second light drive signals together provide a second differential light drive signal to the second light source.

3. The system of claim 2, wherein the first differential light drive signal is 180 degrees out-of-phase with the second differential light drive signal.

4. The system of claim 3:
wherein the system further comprises the first light source and the second light source;
wherein the first light source comprises a first LED light source;
wherein the second light source comprises a second LED light source; and
wherein the first LED light source and the second LED light source are driven in a configuration such that the first LED light source is turned on when the differential light drive signal exceeds an illumination threshold of the first light source, and the second LED light source is turned on when the second differential light signal exceeds an illumination threshold of the second light source.

5. The system of claim 1:
wherein the system further comprises the first light source and the second light source;
wherein the first light source comprises a first LED light source, and the second light source comprises a second LED light source;
wherein the first LED light source and the second LED light source are wired in a back-to-back configuration; and
wherein the first differential light drive signal results in current alternately flowing through the first LED light source and the second LED light source.

6. The system of claim 5, wherein a current associated with the first LED light source is 180 degrees out-of-phase with a current associated with the second LED light source.

7. The system of claim 1, wherein the first differential light drive signal controls the direction of current through the first light source.

8. The system of claim 1, wherein the second light drive signal comprises a DC bias voltage.

9. The system of claim 1, wherein the first and the second light drive signal generators comprise unipolar signal generators.

10. The system of claim 1, further comprising processing equipment configured to:
receive a physiological light signal corresponding to light emitted by the first light source and attenuated by a subject; and
determine a physiological parameter based at least in part on the physiological light signal.

11. The system of claim 10, wherein the physiological parameter is selected from the group consisting of oxygen saturation, pulse rate, respiration rate, respiration effort, blood pressure, hemoglobin concentration, and any combination thereof.

12. A method for driving a light source for a physiological monitor, the method comprising:
generating, using a first light drive signal generator, a first light drive signal to be applied to a first input of a first light source; and
generating, using a second light drive signal generator, a second light drive signal to be applied to a second input of the first light source, wherein the first light drive signal and the second light drive signal together provide a first differential light drive signal to the first light source.

13. The method of claim 12, further comprising generating, using a third light drive signal generator, a third light drive signal to be applied to a first input of a second light source, wherein:
the second light drive signal is further configured to be applied to a second input of the second light source; and
the third and second light drive signals together provide a second differential light drive signal to the second light source.

14. The method of claim 13, wherein the first differential light drive signal is 180 degrees out-of-phase with the second differential light drive signal.

15. The method of claim 14, further comprising
driving the first light source and the second light source in a configuration such that the first light source is turned on when the first differential light drive signal exceeds an illumination threshold of the first light source and the second light source is turned on when the second differential light signal exceeds an illumination threshold of the second light source.

16. The system of claim 12, wherein the first differential light drive signal controls the direction of current through the first light source.

17. The method of claim 12, wherein the second light drive signal comprises a DC bias voltage.

18. The method of claim 12, wherein the first and the second light drive signal generators comprise unipolar signal generators.

19. The method of claim 12, further comprising:
receiving, using processing equipment, a physiological light signal corresponding to light emitted by the first light source and attenuated by a subject; and
determining, using the processing equipment, a physiological parameter based at least in part on the physiological light signal.

20. The method of claim 21, wherein the physiological parameter is selected from the group consisting of oxygen saturation, pulse rate, respiration rate, respiration effort, blood pressure, hemoglobin concentration, and any combination thereof.
